(19) United States
(12) Patent Application Publication
(10) Pub. No.: US 2011/0152794 A1
(43) Pub. Date: Jun. 23, 2011

(54) Throat obstruction dislodging device and method for its use

(76) Inventor: Alan R. Carver, Santa Rosa Beach, FL (US)

(21) Appl. No.: 12/653,645

(22) Filed: Dec. 17, 2009

(51) Int. Cl. G09B 19/24; A61M 1/00

(52) U.S. Cl. 604/275; 434/262

(57) ABSTRACT

A device that helps clear an obstruction lodged in a person's throat has a barrel with a proximal end and an open distal end. A plunger assembly is slidably received within the barrel through the proximal end such that drawing the plunger outwardly from the proximal end produces a suction in the barrel thereby drawing air into the barrel through the distal end. The inside diameter at the distal end is relatively smaller compared to the inside diameter of the barrel at its medial section. One or more O-rings encircle the outer surface of the barrel at the distal end. The distal end is intubated into the throat of the person with the O-ring helping create a seal within the throat. The plunger is drawn outwardly from the proximal end drawing air into the distal end causing a suction in the throat, which suction helps dislodge the obstruction upwardly toward the mouth.

### BACKGROUND OF THE INVENTION

1. Field of the Invention

The present invention relates to a device that is inserted into a person's mouth and helps dislodge an obstruction lodged within the person's throat.

2. Background of the Prior Art

Parents of children, especially young children, tend to have many worries on their minds with regard to their children. From SIDS, to swine flu, to injuries due to falls, to the various ailments that can afflict children, parents' worries can sometimes seem daunting. One major fear for parents of young children is choking. Many young kids, typically those under the age of three, will put most everything and anything into their mouth. While some such items may simply be disgusting, other items, such as small parts can get lodged in a child's throat leading to an obstruction so that the child is unable to breathe possibly leading to death. Manufacturers of toys and other items designed for children, put warning labels onto their products that alert parents to the presence of small parts that can pose a choking hazard. However, it is nearly impossible to keep every small item that is capable of choking a child out of the child's reach. Older children may have toys that have small parts which toys the younger child can easily gain access to, especially if the older child does not clean up after playing—children tend to lack the safety diligence of parents. While playing outside, a small child can easily place a rock or other object into his or her mouthing leading to choking. Even the most vigilant parent cannot maintain constant visual contact with the young child. It only takes a few seconds for a potentially disastrous situation to commence.

While emergency personnel are trained to dislodge an obstruction in a child's throat, or otherwise provide an air passage if the obstruction cannot be readily removed (emergency tracheotomy), brain damage begins after the brain has been deprived of oxygen for about three minutes, faster for younger children. Therefore, unless trained emergency personal are onsite almost immediately after the object lodges in a child's throat, the potential for an adverse outcome is great.

Although the potential for choking tends to be highest among young children, adults are not immune from choking potential. Food going down the throat the wrong way can potentially lead to choking in an adult. If the adult is in a restaurant or other public place, another person can initiate the Heimlich maneuver on the choking person. Even if the person is not properly trained in the Heimlich, a less than perfect attempt can dislodge the obstruction, even if it does cause minor injury to the choking person. After all, a cracked rib is far superior to death. However, in a relatively secluded place, such as a person's home where the person may be at home alone or only with his or her spouse, improperly lodged food has greater potential for choking. A person alone cannot perform the Heimlich on themselves. If a spouse is present and is either too feeble or too scared to perform the Heimlich, the next best resort is summoning emergency personal. As noted above, such summoning may not prove adequate.

What is needed is a device that can quickly dislodge an obstruction from a person's throat in order to prevent choking. Such a device must be very easy to use so that a person who has not used the device before and who may be in a panic, can quickly employ the device in order to remove the obstruction and potentially save a life. Ideally, such a device will be of relatively simple design and construction so as to be affordable to a large portion of the population.

### SUMMARY OF THE INVENTION

The throat obstruction dislodging device of the present invention addresses the aforementioned needs in the art by providing a device that can be quickly deployed in the case of a throat obstruction and can remove the obstruction in order to prevent choking. The throat obstruction dislodging device is designed to be nearly foolproof so that a person who is unfamiliar with the device can still employ the device effectively in a very rapid amount of time. A choking person can use the device on himself or herself. The throat obstruction dislodging device is of relatively simple design and construction so as to be relatively inexpensive to produce so that it is relatively affordable to a large segment of the population.

The throat obstruction dislodging device is comprised of a barrel that has an open proximal end, an open distal end, a medial section, and an outer surface. A plunger assembly is slidably disposed within the proximal end such that when the plunger is drawn outwardly toward the proximal end, a suction is created within the barrel and air is drawn into barrel through the distal end. An O-ring encircles the outer surface of the barrel at the distal end. The distal end of the barrel has a first inside diameter and the medial section of the barrel has a second inside diameter that is greater than the first inside diameter. A face plate is attached to the outer surface of the barrel at the medial section. The face plate is symmetrical along a vertical midline and has a pair of curved section, one curved section on each side of the vertical midline, the face plate also having a top edge and a cutaway section straddling the vertical midline.

### BRIEF DESCRIPTION OF THE DRAWINGS

FIG. 1 is an environmental view of the throat obstruction dislodging device of the present invention being used on a person with an obstruction.

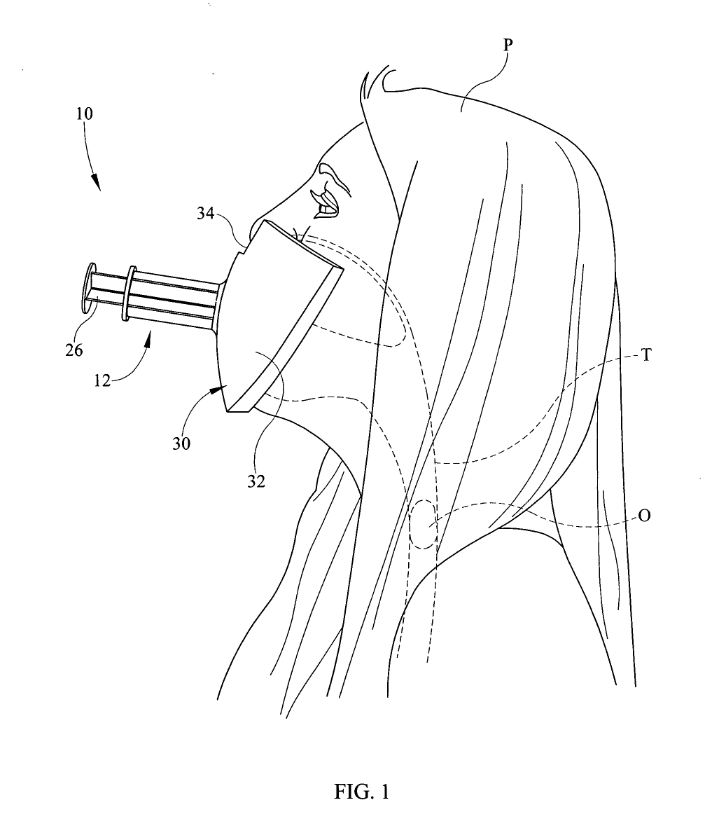

FIG. 2 is a perspective view (front) of the throat obstruction dislodging device.

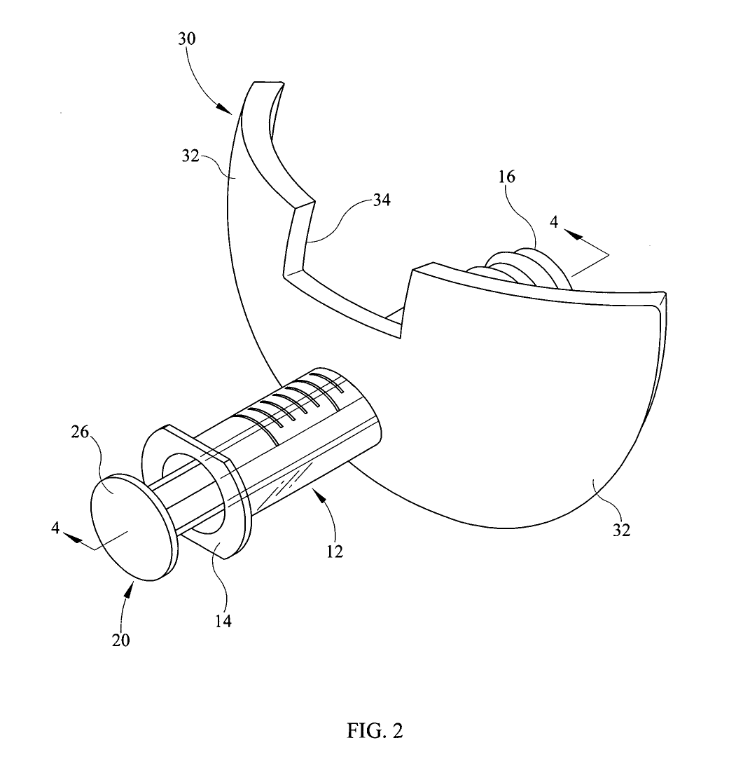

FIG. 3 is a perspective view (rear) of the throat obstruction dislodging device.

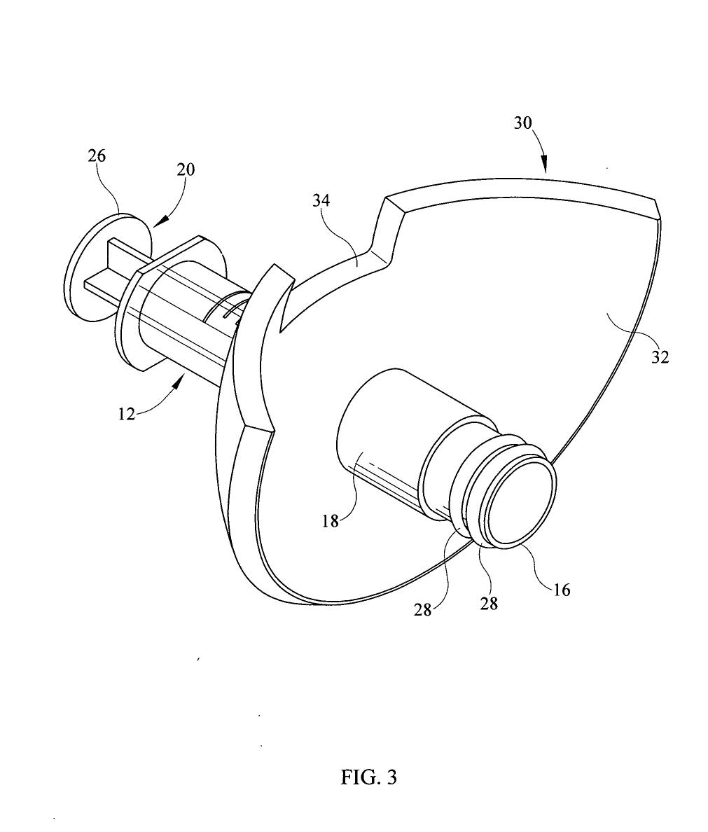

FIG. 4 is a sectioned view of the throat obstruction dislodging device.

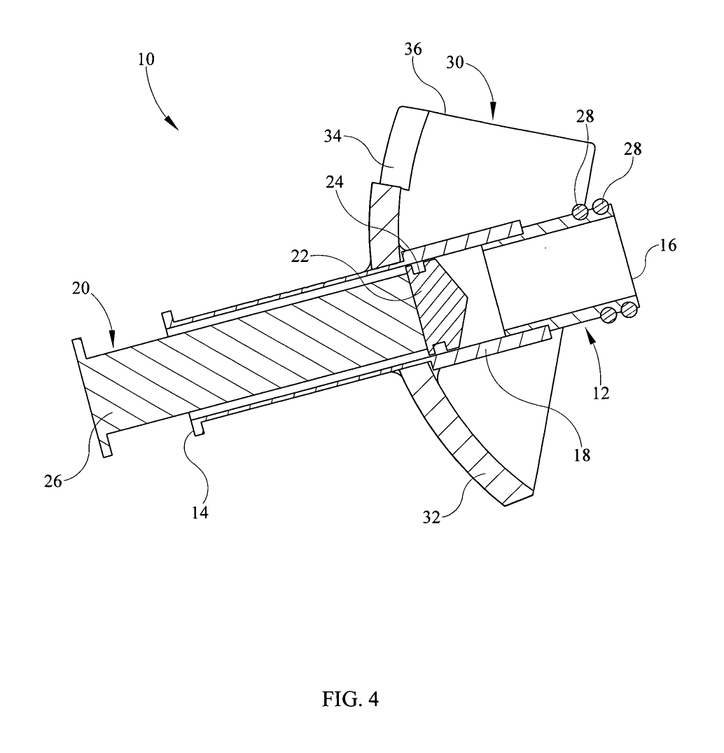

Similar reference numerals refer to similar parts throughout the several views of the drawings.

### DESCRIPTION OF THE PREFERRED EMBODIMENT

Referring now to the drawings, it is seen that the throat obstruction dislodging device of the present invention, generally denoted by reference numeral 10, is comprised of a barrel 12 that has a proximal end 14, a distal end 16, each of which is open, and a medial section 18. The barrel 12 is made from an appropriate plastic material, preferably a bioacceptable plastic material. As seen, the medial section 18 has a relatively larger inside diameter compared to the inside diameter of the distal end 16 in order to gain more velocity in the air moved by the device 10 during use as more fully explained below, although a barrel 12 that has a uniform inside diameter can also be used. A plunger 20 is slidably disposed through the proximal end 14 of the barrel 12, the plunger 20 having a piston 22 that has a rubber head 24 on its end, which rubber head has an annular ring, the rubber head 24 creating a relatively airtight fit within the barrel 12. The plunger 20 also has a handle 26. Drawing the plunger 20 from the distal end 16 to the proximal end 14 creates a suction within the barrel 12 thereby drawing air through the distal end 16. One or more O-rings 28 encompass the outer surface of the barrel 12 at its distal end 16. These O-rings 28 are made from a resilient material, preferably a bioacceptable material, such as rubber or neoprene.

A face plate 30 is attached to an outer surface of the barrel 12 at the medial section 18 in encircling fashion and is held thereat in appropriate fashion (adhesion, ultrasonic welding, etc.). As seen, the face plate 30 is substantially symmetrical on its vertical midline and has a pair of curved portions 32 on either side of the midline and has a cutaway section 34 along its top edge 36 at the midline.

In order to use the throat obstruction dislodging device 10 of the present invention, the plunger 20 is pushed in as far as possible toward the distal end 16. The device 10 is placed onto the face of a person P that has an obstruction O lodged in his or her throat T. The face plate 30 helps with quick and proper positioning such that the person's cheeks are received within the curved portions 32 of the face plate 30 and the person's nose is within the cutaway section 34 of the face plate 30. The face plate 30 also acts as a stop and helps prevent over insertion of the barrel 12 into the throat T. In this position, the distal end 16 of the barrel 12 is intubated within the throat T of the person with the O-rings 28 encompassing the outer surface of the barrel 12 helping create a seal between the barrel 12 and the throat T. The plunger 20 is drawn outwardly away from the distal end 16 such that the piston 22 creates a suction within the barrel 12 which causes air to be drawn into the barrel 12 via the distal end 16 which air is drawn upward through the throat T. This drawn air creates a suction within the throat which suction helps draw the obstruction O up and out of the throat T. The use of a staged inside diameter within the barrel 12 helps create a relatively higher air velocity of air movement at the distal end 16 when the piston 22 of the plunger 20 is being drawn toward the proximal end 14 which increased velocity further helps dislodge the obstruction O. For a particularly deep lodged obstruction O, a second iteration of use of the device 10 may be needed with the device 10 being removed from the person's throat T and mouth prior to depressing the plunger 20 back into the barrel 12.

The throat obstruction dislodging device 10 may come in various sizes for various sized people P such as a child sized device 10 and an adult sized device 10 with the size and length of the barrel 12 and the size of the face plate 30 being adjusted accordingly (of course, more than two sizes are possible).

As an additional option, an electronic sound producing device (not illustrated) can be located on the face plate 30 (or possibly the barrel 12), which sound producing device has a prerecorded message thereon (on a memory chip) such that the pressing of a button triggers activation of the sound producing device which produces the message through a speaker (piezo-electric speaker, or similar), which message is instructions on proper use of the device 10 so that a person not familiar with the device presses the activation button and listens to the instruction on proper device 10 usage, which instructions can be paused or replayed s needed. Of course a small written message is placed on the face plate 30 instructing a user to depress the activation button in order to receive the instructional message.

While the invention has been particularly shown and described with reference to an embodiment thereof, it will be appreciated by those skilled in the art that various changes in form and detail may be made without departing from the spirit and scope of the invention.

I claim:

1. A suction device comprising:
a barrel having an open proximal end, an open distal end, a medial section therebetween, and an outer surface;
a plunger assembly slidably disposed within the proximal end such that when the plunger is drawn outwardly through the proximal end, a suction is created within the barrel and air is drawn into barrel through the distal end; and
an O-ring encircling the outer surface of the barrel at the distal end.

2. The suction device as in claim 1 wherein the distal end of the barrel has a first inside diameter and the medial section of the barrel has a second inside diameter that is greater than the first inside diameter.

3. The suction device as in claim 2 further comprising a face plate attached to the outer surface of the barrel at the medial section.

4. The suction device as in claim 3 wherein the face plate is symmetrical along a vertical midline and has a pair of curved section, one curved section on each side of the vertical midline, the face plate also having a top edge and a cutaway section straddling the vertical midline.

5. The suction device as in claim 3 further comprising a sound producing device attached to the face plate or the barrel for producing an instructional audio message on use of the suction device.

6. The suction device as in claim 1 further comprising a face plate attached to the outer surface of the barrel at the medial section.

7. The suction device as in claim 6 wherein the face plate is symmetrical along a vertical midline and has a pair of curved section, one curved section on each side of the vertical midline, the face plate also having a top edge and a cutaway section straddling the vertical midline.

8. The suction device as in claim 6 further comprising a sound producing device attached to the face plate or the barrel for producing an instructional audio message on use of the suction device.

9. The suction device as in claim 1 further comprising a sound producing device attached to the barrel for producing an instructional audio message on use of the suction device.

10. A method for dislodging an obstruction lodged within a throat of a mammal, the method comprising the steps of:
providing a barrel having an open proximal end, an open distal end, a medial section, and an outer surface;
providing a plunger assembly and slidably disposing the plunger assembly within the proximal end such that when the plunger is drawn outwardly through the proximal end, a suction is created within the barrel and air is drawn into barrel through the distal end;
providing an O-ring and encircling the O-ring about the outer surface of the barrel at the distal end;
inserting the distal end of the barrel through a mouth of the mammal into the throat; and
drawing the plunger assembly outwardly through the proximal end.

11. The method as in claim 10 wherein the distal end of the barrel has a first inside diameter and the medial section of the barrel has a second inside diameter that is greater than the first inside diameter.

12. The method as in claim 11 further comprising the steps of:
providing a face plate attached to the outer surface of the barrel at the medial section; and
placing the face plate onto a face of the mammal

13. The method as in claim 12 wherein the face plate is symmetrical along a vertical midline and has a pair of curved section, one curved section on each side of the vertical midline, the face plate also having a top edge and a cutaway section straddling the vertical midline.

14. The method as in claim 12 further comprising the steps of:
producing a sound producing device that produces an instruction audio message; and
attaching the sound producing device onto the barrel or onto the face plate.

15. The method as in claim 10 further comprising the steps of:
providing a face plate attached to the outer surface of the barrel at the medial section; and
placing the face plate onto a face of the mammal

16. The method as in claim 15 wherein the face plate is symmetrical along a vertical midline and has a pair of curved section, one curved section on each side of the vertical midline, the face plate also having a top edge and a cutaway section straddling the vertical midline.

17. The method as in claim 15 further comprising the steps of:
producing a sound producing device that produces an instruction audio message; and
attaching the sound producing device onto the barrel or the face plate.

18. The method as in claim 10 further comprising the steps of:
producing a sound producing device that produces an instruction audio message; and
attaching the sound producing device onto the barrel.
